(19) United States
(12) Patent Application Publication
(10) Pub. No.: US 2022/0088414 A1
(43) Pub. Date: Mar. 24, 2022

(54) TREATMENT RISK MITIGATION

(71) Applicant: International Business Machines Corporation, Armonk, NY (US)

(72) Inventors: Zachary A. Silverstein, Jacksonville, FL (US); Kelley Anders, East New Market, MD (US); Jonathan D. Dunne, Dungarvan (IE); Liam S. Harpur, Dublin (IE)

(21) Appl. No.: 17/029,152

(22) Filed: Sep. 23, 2020

(51) Int. Cl. A61N 5/10; G16H 70/20; G16H 50/20; G16H 50/70; G16H 50/30; G16H 40/67; G16H 20/40

(52) U.S. Cl. CPC A61N 5/1067; G16H 70/20; G16H 50/20; G16H 50/70; A61N 5/1071; G16H 40/67; G16H 20/40; A61N 5/1038; G16H 50/30

(57) ABSTRACT

A set of baseline treatment information is received. From the set of baseline treatment information, a set of incidental information is determined. Based on the set of baseline treatment information and the set of incidental information, a treatment mitigation area is generated. Based on the set of baseline treatment information, the set of incidental information, and the treatment mitigation area, a treatment mitigation model is created. An affected individual is identified, based on the treatment mitigation model. The affected individual is notified of one or more treatment mitigation recommendations.

### BACKGROUND

The present disclosure relates generally to the field of patient treatment plans, and more particularly to mitigating treatment risk.

Certain medical treatments can cause exposure risks to patients and other persons with whom patients come into contact. For example, chemotherapy and radiation-based treatments can create significant exposure risks. In such cases, relatives and caretakers for the patient, as well as members of the general public, may, knowingly or unknowingly, receive a certain amount of exposure.

### SUMMARY

Embodiments of the present disclosure include a method, computer program product, and system for mitigating treatment risk.

A set of baseline treatment information is received. From the set of baseline treatment information, a set of incidental information is determined. Based on the set of baseline treatment information and the set of incidental information, a treatment mitigation area is generated. Based on the set of baseline treatment information, the set of incidental information, and the treatment mitigation area, a treatment mitigation model is created. An affected individual is identified, based on the treatment mitigation model. The affected individual is notified of one or more treatment mitigation recommendations.

The above summary is not intended to describe each illustrated embodiment or every implementation of the present disclosure.

### BRIEF DESCRIPTION OF THE DRAWINGS

The drawings included in the present disclosure are incorporated into, and form part of, the specification. They illustrate embodiments of the present disclosure and, along with the description, serve to explain the principles of the disclosure. The drawings are only illustrative of typical embodiments and do not limit the disclosure.

FIG. 1 illustrates a block diagram of an example high level architecture of a system for mitigating treatment risk, in accordance with embodiments of the present disclosure.

FIG. 2 illustrates an example geofence diagram, in accordance with embodiments of the present disclosure.

FIG. 3 illustrates an example method for mitigating treatment risk, in accordance with embodiments, of the present disclosure

FIG. 4 depicts an example neural network for identifying treatment mitigation model adjustments, in accordance with embodiments of the present disclosure.

FIG. 5 depicts a cloud computing environment according to an embodiment of the present disclosure.

FIG. 6 depicts abstraction model layers according to an embodiment of the present disclosure.

FIG. 7 depicts a high-level block diagram of an example computer system that may be used in implementing embodiments of the present disclosure.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the disclosure.

### DETAILED DESCRIPTION

Aspects of the present disclosure relate generally to the field of patient treatment plans, and more particularly to mitigating treatment risk. While the present disclosure is not necessarily limited to such applications, various aspects of the disclosure may be appreciated through a discussion of various examples using this context.

Certain medical treatments can cause exposure risks to patients and other persons with whom patients come into contact. In such circumstances, a treated patient may pose a degree of risk to their environment. For example, chemotherapy and radiation-based treatments can create significant exposure risks; and some medications/treatments must be handled with care to avoid contamination of an area and/or administering individual. In such cases, relatives and caretakers for the patient, as well as members of the general public, may, knowingly or unknowingly, receive a certain amount of exposure to a treatment and/or harmful condition.

Ideally, individuals affected by an exposure would take steps to mitigate the effects of the exposure. However, many such individuals (and especially those of the general public) may not realize they have been exposed, or may not know what the proper mitigation steps include. In some circumstances, multiple exposures to multiple different treatments may require additional mitigation. In yet other circumstances, a patient receiving a particular treatment should be kept from interacting with another patient receiving another treatment, whether that be the same treatment, or a different one.

Embodiments of the present disclosure contemplate the generation of a treatment mitigation model by ingesting information regarding treatments (e.g., baseline information) the risk and mitigation steps, as well as any related information, for various treatments (e.g., incidental information), as well as information regarding the individual patients, caretakers, and members of the general public who may opt in, and/or opt out, to using and/or participating in such a model (e.g., demographic and behavioral/historical information). Such information may populate a table, text index, relational database, or other suitable data structure, to generate a rules-based, or other transparent model. Any information handled by embodiments of the present disclosure shall be stored securely and in accordance with the relevant privacy laws (e.g., HIPAA, GDPR, etc.) and regulations.

In some embodiments, such a transparent model approach may be preferable over a separate neural networking approach (e.g., non-transparent), as it provides clarity regarding tailored mitigation steps that should be taken for each treatment, according to each individual. In this way, a potentially affected individual may reap significant benefits from an automatically generated and more comprehensive record of potential exposures, which may be provided to a medical professional or care provider, who in turn may provide recommendations, oversight, and/or adjustments to the model, as needed.

In some embodiments, individuals who participate in using the treatment mitigation model may be monitored to determine individual outcomes, and this data, as well as the data within the treatment model itself, may be further ingested by a separate neural network in order to identify trends and make predictions regarding the effectivity of mitigation recommendations/steps. In such embodiments, the neural network may provide suggestions for adjustments to the relevant medical professional and, potentially, the treatment mitigation model. In other embodiments, the adjustment suggestions for the model may be incorporated automatically, provided a set of criteria are met (e.g., predicted change in effectivity and overall risk meets or exceeds a threshold/parameter).

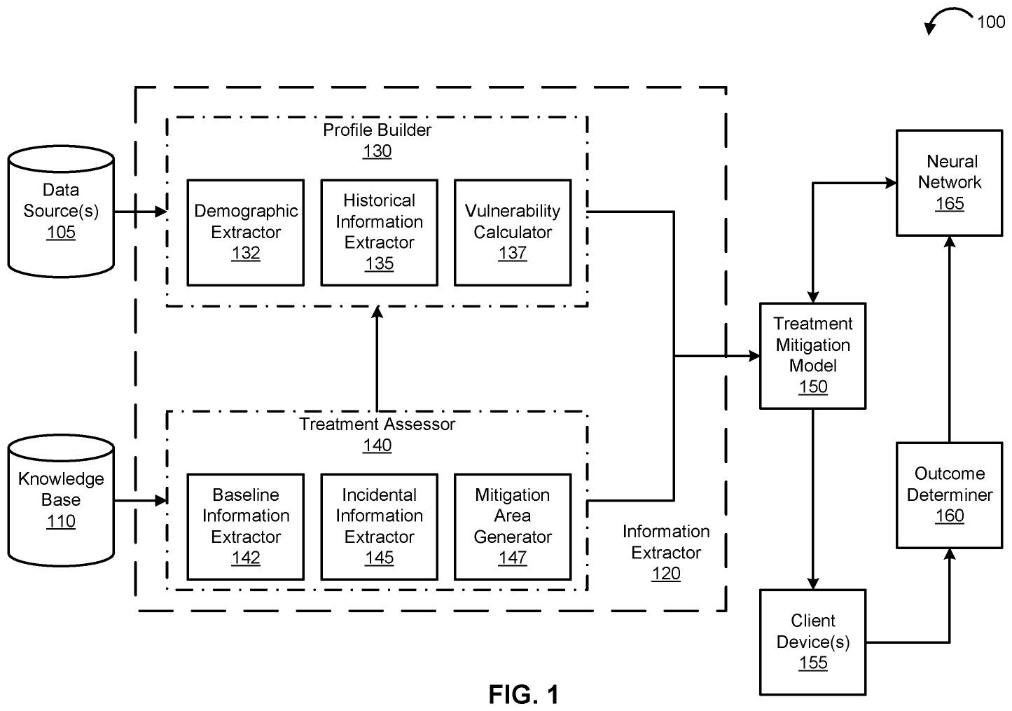

Turning now to FIG. 1, illustrated is a block diagram of an example high level architecture of a system for mitigating treatment risk 100, in accordance with embodiments of the present disclosure. The system for mitigating treatment risk 100 may include, for example, data source(s) 105, knowledge base 110, information extractor 120, treatment mitigation model 150, client device(s) 155, outcome determiner 160, and neural network 165.

The various components of the system for mitigating treatment risk 100 may by distant from each other and may communicate over a network (not pictured). In some embodiments, such a network can be implemented using one or more client-server models, peer-to-peer configurations, or any other suitable network topology. For example, such a network may be a wide area network (WAN), a local area network (LAN), the Internet, or an intranet. In some embodiments, a subset of the components (e.g. the components of information extractor 120) may be local to each other and communicate via any appropriate local communication medium. For example, such a subset of components may communicate using a LAN, one or more hardwire connections, a wireless link or router, or an intranet.

In some embodiments, the various components of the system for mitigating treatment risk 100 may be communicatively coupled using a combination of one or more networks and/or one or more local connections. For example, the components of the information extractor 120 may be hardwired to each other, while the remainder of the system components may communicate using a network (e.g., over the Internet).

In some embodiments, the various components of the system for mitigating treatment risk 100 may be communicatively coupled within a cloud computing environment, or using one or more cloud computing services. Consistent with various embodiments, a cloud computing environment may include a network-based, distributed data processing system that provides one or more cloud computing services. Further, a cloud computing environment may include many computers (e.g., hundreds or thousands of computers or more) disposed within one or more data centers and configured to share resources over such a network.

In some embodiments, information extractor 120 may include, for example, a profile builder 130 and a treatment assessor 140. Profile builder 130 may further include a demographic extractor 132, a historical information extractor 135, and a vulnerability calculator 137.

Treatment assessor 140 may receive or retrieve, using baseline information extractor 142, baseline information regarding various treatments associated with exposure risks from knowledge base 110. Knowledge base 110 may, in some embodiments, include multiple sources (e.g., repositories at the Center for Disease Control, the National Institutes of Health, a state or county board of health, a hospital or proprietary database, etc.). Baseline information extractor 142 may, for example, retrieve a set of baseline information for treatments with radioactive exposure risks, chemical exposure risks, etc. In some embodiments, baseline information may include information from standard operating procedure (SOP) manuals, medical journals, material safety data sheets (MSDS), etc.

In some embodiments, incidental information extractor 145 may leverage the retrieved baseline information to search for, and retrieve/extract relevant information that may not be natively found within the baseline information itself. For example, a chemotherapeutic treatment may mention iodine-131, but it may lack further information regarding the radioactive qualities of iodine-131, such as area of affect, radioactive half-life, biological half-life, radioactive intensity, radiation type, clearance rate, exposure rate, total amount of exposure, etc. Incidental information extractor 145 may search additional sources within knowledge base 110 to extract such incidental information, if available. Information regarding mitigation for a particular treatment may be identified in either baseline or incidental information, depending upon the embodiment. In some embodiments, incidental information may include a caution score for a particular treatment, which may reflect the level of risk a treatment poses to a patient or other individual (e.g., total exposure amount, exposure rate, etc.). For example, a caution score may take into account MSDS information, biohazard information, radiological information, etc.

In some embodiments, mitigation area generator 147 may analyze the baseline information and the incidental information to generate a treatment mitigation area. In some embodiments, a treatment mitigation area may be implemented as a geofence, and may include the radius from a treated patient, or a radius from an area where one or more treatments are regularly performed or where a particular treatment material (e.g., radioactive isotopes, biohazardous chemicals, etc.) is stored. A treated patient may be, after opting in and in some embodiments, tracked using a mobile device, such as a smart phone, smart watch, or any other global positioning system (GPS) enabled device.

In some embodiments, the treatment mitigation area may represent an area that, were an individual to enter, such an individual should either take a precautionary measure (e.g., wearing appropriate personal protective equipment (PPE)) or take a mitigation step after the fact (e.g., washing hands, showering, avoiding a second exposure for X amount of time, etc.). In some embodiments, such as when two treated patients are nearing each other, the treatment mitigation area may be used to warn each treated patient of the presence and direction of the potential exposure.

Profile builder 130 may receive or retrieve, via demographic extractor 132, demographic information for participating individuals (e.g., patients, caretakers, care providers, member of the public, and any other individual that has opted into using the system for mitigating treatment risk 100). This may include prompting a user (e.g., participating individual) to fill out a form, or it may include retrieving information from a linked or preexisting source of information, such as data source(s) 105. In some embodiments, data source(s) 105 may include medical records (so long as an appropriate release has been obtained), social media accounts, public records, etc.

Profile builder 130 may further receive or retrieve, via historical information extractor 135, historical information regarding a user. Historical information may include, for example, location history, behavioral traits/patterns, medications taken, treatments received, medical conditions, etc. In some embodiments, the user may be prompted to provide a signed release for any information protected or regulated by law. Historical information extractor 135 may, in some embodiments, retrieve such information from one or more data source(s) 105, or it may infer such information based on information extracted by demographic extractor 132 in conjunction with a portion of retrieved historical information.

Vulnerability calculator 137 may determine, based on the demographic information and the historical/behavioral information, a vulnerability score for a participating individual. In some embodiments, a vulnerability score may be a generalized score for all treatments.

In some embodiments, profile builder 130 may receive, from treatment assessor 140, information relevant to generating a specialized vulnerability score. For example, a particular user's vulnerability score may change, depending on the type and duration of a potential exposure. As such, baseline information, incidental information, and treatment area may all be factored into a vulnerability score for a particular user.

In some embodiments, a user profile may contain multiple vulnerability scores. For example, a single user may have a general vulnerability score as well as one or more specialized vulnerability scores for when particular exposure types are in play. For example, a patient receiving chemotherapy may have a greater vulnerability to secondary radiation exposure, but a lesser vulnerability when exposed to certain chemical treatments. In this way, vulnerability may be assessed according to both user profile and exposure type.

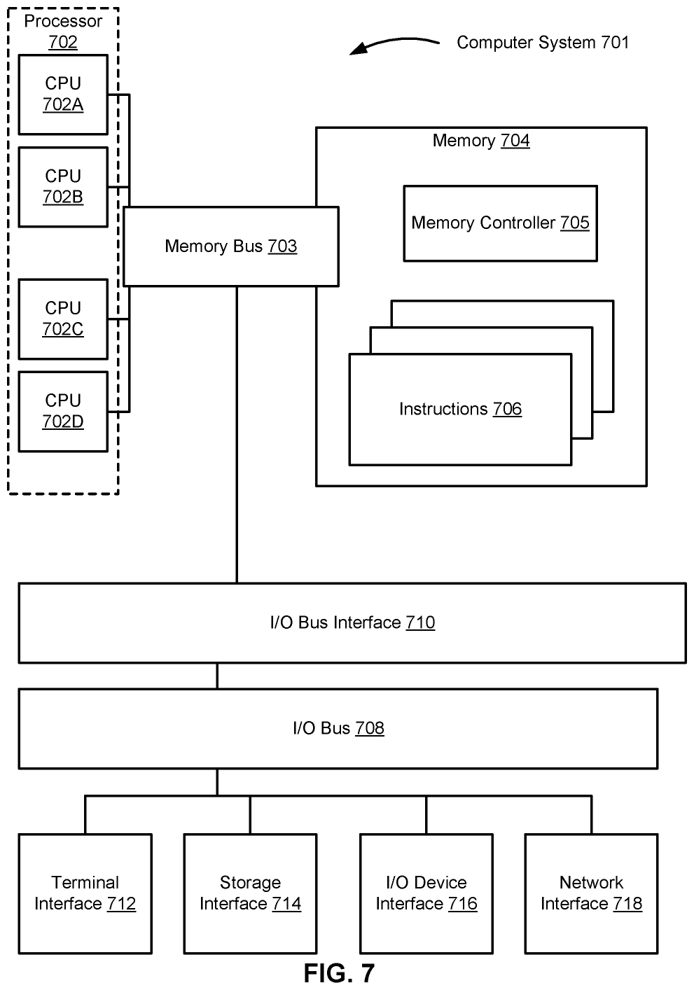

Treatment mitigation model 150 may receive the information extracted and/or generated by both profile builder 130 and treatment assessor 140. Treatment mitigation model 150 may store such information in any suitable data structure, such as a table or relational database (not pictured). Such a data structure may be stored, for example, using storage interface 714 of FIG. 7.

TABLE 1
Table 1 depicts an example (not real data) of a table that could be stored in a
database or other suitable data structure used by treatment mitigation model 150:

| | Patient | | | | Amount | | | |
|---|---|---|---|---|---|---|---|---|
| Treatment | demographics | Decay | Intensity | | of | | . . . N | |
| Type | components | rate | (in Grays) | | Exposure | | component(s) | Action |
| Chemo- | Age: 80, Sex: F, | 2 | 4 | mcg | 8 | mcg | . . . | Wear |
| Cisplatin | Height: 5′8″, | weeks | | | | | | protective |
| | Weight: 180 lbs | | | | | | | clothing; if |
| | | | | | | | | sweating |
| | | | | | | | | profusely |
| | | | | | | | | within 2 |
| | | | | | | | | weeks after |
| | | | | | | | | treatment, |
| | | | | | | | | wash |
| | | | | | | | | clothes |
| | | | | | | | | separately. |
| Proton- | Age: 80, Sex: F, | 2 | .75 | sieverts | 5 | sieverts | . . . | No action |

| | | | | | | |
|---|---|---|---|---|---|---|
| Therapy | Height: 5′8″, | hours | | | | needed, no |
| | Weight: 180 lbs | | | | | radiation |
| | | | | | | risk for |
| | | | | | | others |
| . . . N | . . . | . . . | . . . | . . . | . . . | . . . |
| treatment | | | | | | |

Treatment mitigation model 150 may provide information regarding the mitigation of treatment risks to a medical professional for review/approval, and subsequently to other participating individuals. For example, treatment mitigation model may send, to a client device(s) 155 associated with such a medical professional, individualized mitigation plans detailing precautionary steps for avoiding exposure risks. In addition, treatment mitigation model 150 may, in some embodiments, use geofences and real-time position monitoring of client device(s) 155 to prevent the collision of treatment mitigation areas, or to warn users (e.g., after review by a medical professional) when they have entered a treatment mitigation area, and to inform users of any additional mitigation steps, which may be recommended by the medical professional, that the user may wish to perform in order to reduce their exposure risk.

In some embodiments, treatment mitigation model 150 may leverage the predictive capabilities of a neural network 165 to anticipate the travel path of a user and preemptively warn them of a potential exposure. In some embodiments, treatment mitigation model 150 may provide a user with a general area risk score, which may represent the risk of exposure in a particular area (e.g., a city park, a shopping mall, a hospital, etc.).

In embodiments where users have opted into outcome monitoring, the client device(s) 155 associated with such users may be monitored by outcome determiner 160. Outcome determiner 160 may track such users' activities (e.g., travel logs, exercise journal, diet, medications, medical checkups, interactions with other users, duration of activities, etc.) to provide outcome datasets which may be analyzed by the model or separate neural network, and may be subsequently reviewed by a medical professional, to identify adjustments that could be made to improve the accuracy and efficacy of treatment mitigation model 150.

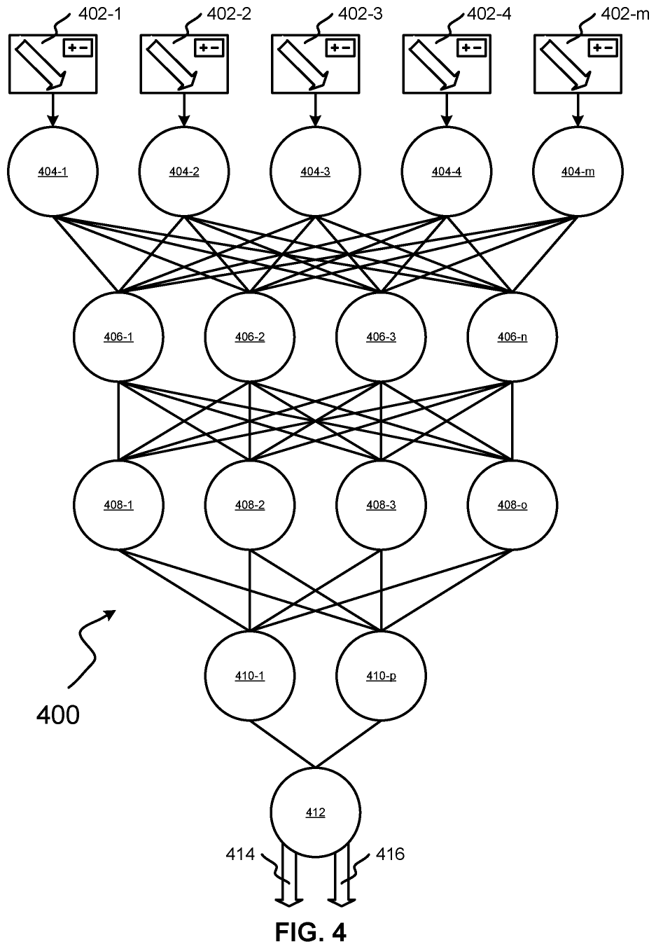

In some embodiments, a neural network 165 may be leveraged to identify trends among treatments/exposures, user profile information, user activities, and user outcomes. In such an embodiment, neural network 165 may automatically adjust treatment mitigation model 150, or it may generate recommendation for review and approval by medical experts and developers/administrators of treatment mitigation model 150. Additional detail regarding the operation of neural network 165 is given with regard to FIG. 4.

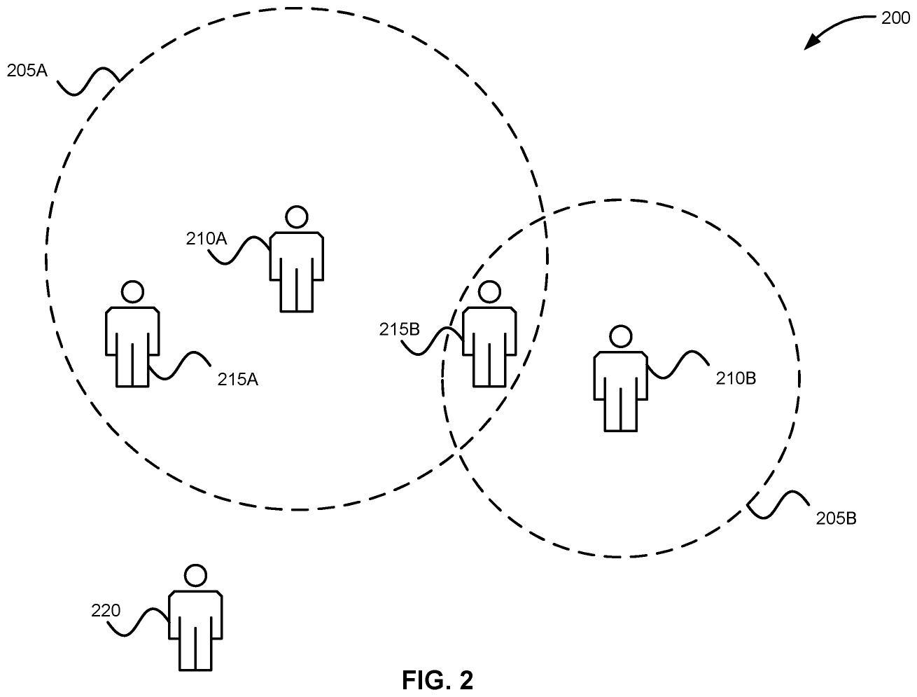

Turning now to FIG. 2, illustrated is an example geofence diagram 200, in accordance with embodiments of the present disclosure. Example geofence diagram 200 includes treatment mitigation areas 205A-B, and various participating users, such as treated patients 210A-B, care providers 215A-B, and the general public 220.

In this example, treated patient 210A may have received a treatment that carries an exposure risk to others, such as chemotherapy. Treatment mitigation area 205A represents the area in which an individual would be considered “exposed.” As such, care providers 215A-B, in this example, would be considered as receiving exposure to whatever treatment treated patient 210A had received. In some embodiments, care providers 215A-B may have knowingly been exposed during the administration of the treatment, or it may have been an inadvertent exposure. Treatment mitigation model 150 may, for example, automatically send mitigation information to the appropriate medical professional in charge of reviewing mitigation not only for treated patient 210A, but also for care providers 215A-B. In this way, the appropriate medical professional may notify them of the exposure, and send them mitigation information, as well. In some embodiments where the care providers are previously associated with treated patient 210A, the knowledge of exposure may be assumed, and the notification may be reported as part of a daily/weekly/monthly summary of exposure.

Further, in this example, treated patient 210B may have also received a treatment with exposure risk. For example, patient 210B may have received a topical treatment with a volatile chemical compound that should be used only in well-ventilated areas. Treatment mitigation area 210B reflects the area in which a user would be considered “exposed” to the volatile chemical, and therefore may need a notification, for example, from the appropriate medical professional, reminding them to get fresh air, turn on an air circulation device, or potentially even shower after the exposure. In embodiments where preemptive measures are sent to users, a user may be reminded to avoid wearing contact lenses, for example, prior to entering treatment mitigation area 205B.

In this example, care provider 215B may be considered “exposed” to both treatments and may therefore receive both sets of mitigation information and, prior to the exposures, any preemptive measure information. In some embodiments, the combined exposure from entering both treatment areas 210A-B may call for additional preemptive measures and/or mitigation steps (e.g., showering for X amount of time, using Y type of soap/detergent, avoidance of Z types of exposures for a particular length of time, etc.).

In this example, the general public 220 may receive, via the appropriate medical professional, preemptive measure recommendations if they are predicted to enter either treatment mitigation area 210A or 210B, or if they come into a threshold proximity. If the general public enters either treatment area 210A-B, they may be notified of the appropriate mitigation steps, as dictated by the treatment mitigation model 150 and reviewed/adjusted by the appropriate medical professional.

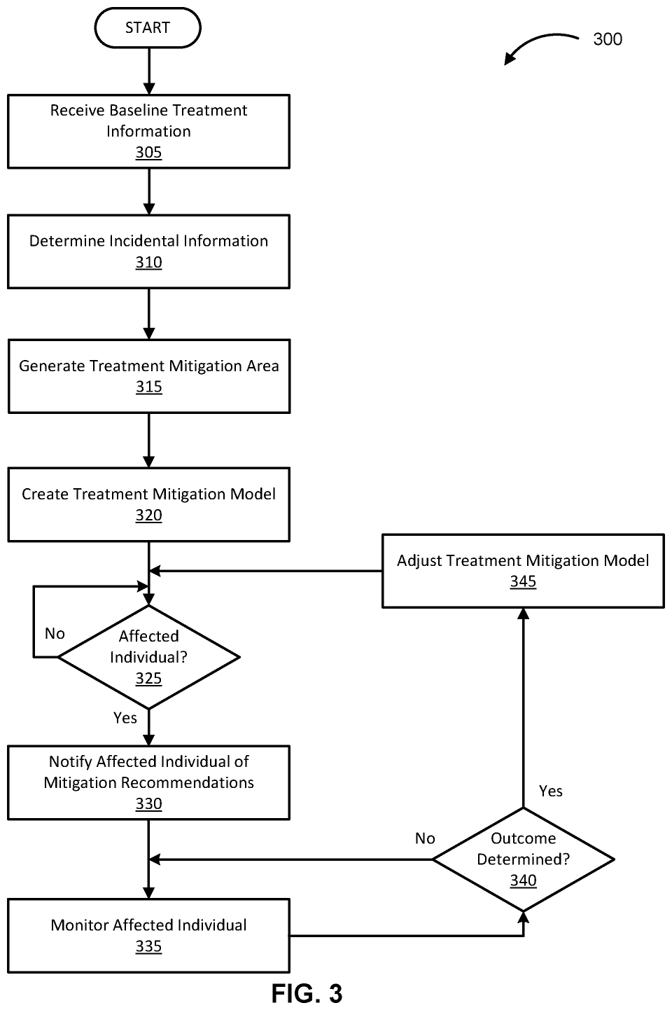

Turning now to FIG. 3, illustrated is an example method 300 for mitigation treatment risk, in accordance with embodiments of the present disclosure. Example method 300 may begin at 305, where baseline treatment information is received. Baseline treatment information may include, for example, treatment type, medication involved, potential exposure type, treatment duration, treatment schedule, treatment intensity (e.g., dose), exposure intensity (e.g., radiation intensity, chemical biohazard level, etc.), etc., as described herein.

At 310, incidental information is determined, based on the baseline treatment information. Incidental information may include, for example, additional information relevant to determining the risk of treatment that may not be apparent from the baseline information. For example, the baseline information may indicate treatment with a chemotherapeutic agent, but it may lack incidental information regarding radiation type (e.g., alpha, beta, gamma), preemptive measures (e.g., what type of PPE to wear), and mitigation measures (e.g., washing with soap and water, preventing further exposures, detoxification medications, etc.).

At 315, a treatment mitigation area is generated. A treatment mitigation area may be based on the baseline and incidental information sets, and may be tailored to reflect a particular treatment and, in some embodiments, the environment in which the treatment was administered (e.g., hospital vs. at home, ventilated area vs closed, shielded vs unshielded, etc.). In some embodiments, the treatment mitigation area may be embodied as a geofence centered on a treated individual or an area in which treatment occurs or treatment materials are stored.

At 325, it is determined whether an affected individual has been identified. In some embodiments, an affected individual may be a treated patient, a caretaker, a care provider (e.g., treatment administrator), or a member of the general public who has opted into participating in the example method 300. Identifying an affected individual may include a determination that the affected individual has crossed a geofence and entered into a treatment mitigation area, as described herein.

If, at 325, no affected individual has been identified, the example method may continue to monitor for affected individuals. If, however, an affected individual is identified at 325, the affected individual is notified of the appropriate mitigation recommendation at 330. This may occur after, for example, the appropriate medical professional has reviewed and approved the recommendation.

At 335, the affected individual may be monitored. This may include location tracking, determining whether the affected individual complied with the mitigation recommendation, monitoring diet, physical activity, duration of various activities, other medications taken, other treatments or medical care received, etc.

At 340, it may be determined whether an outcome has been achieved. In some embodiments, an outcome may be achieved when it has been confirmed that the user did, or did not, comply with the mitigation recommendation. In other embodiments, the outcome may be determined after a set period of time after the mitigation recommendation has been sent to the affected individual. In yet other embodiments, the outcome may be determined multiple times and continuously updated.

If, at 340, an outcome has not been determined, the affected individual may continue to be monitored at 335. If, however, an outcome is determined at 340, the example method 300 may proceed to 345, where an adjustment to the treatment mitigation model is made, based on the determined outcome.

In some embodiments, the adjustment may be determined by identifying trends using a neural network, or it may be determined by medical experts, as described herein. In some embodiments, the adjustment may be automatic. In some embodiments, an automatic adjustment may be allowed only when certain criteria are met, and in yet other embodiments, the adjustments may be sent, in the form of a set of recommendations, to a medical expert or model administrator for review and/or confirmation.

In some embodiments, users participating in method 300 may opt out of participation at any time. In such embodiments, the method may terminate.

FIG. 4 depicts an example neural network 400 that may be used to perform predictions based on, and adjustments to, a treatment mitigation model, in accordance with embodiments of the present disclosure. The example neural network 400, in some embodiments, may be implemented as part of a system for mitigating treatment risk (e.g., system for mitigating treatment risk 100). In some embodiments, parallel techniques (e.g., Single Instruction Multiple Data (SIMD) techniques) may be employed to concurrently adjust multiple data fields within a treatment mitigation model (e.g., treatment mitigation model 150).

In embodiments, neural network 400 may be a classifier-type neural network. Neural network 400 may be part of a larger neural network (e.g., may be a sub-unit of a larger neural network). For example, neural network 400 may be nested within a single, larger neural network, connected to several other neural networks, or connected to several other neural networks as part of an overall aggregate neural network.

Inputs 402-1 through 402-m represent the inputs to neural network 400. In this embodiment, 402-1 through 402-m do not represent different inputs. Rather, 402-1 through 402-m represent the same input that is sent to each first-layer neuron (neurons 404-1 through 404-m) in neural network 400. In some embodiments, the number of inputs 402-1 through 402-m (i.e., the number represented by m) may equal (and thus be determined by) the number of first-layer neurons in the network. In other embodiments, neural network 400 may incorporate 1 or more bias neurons in the first layer, in which case the number of inputs 402-1 through 402-m may equal the number of first-layer neurons in the network minus the number of first-layer bias neurons. In some embodiments, a single input (e.g., input 402-1) may be input into the neural network. In such an embodiment, the first layer of the neural network may comprise a single neuron, which may propagate the input to the second layer of neurons.

Inputs 402-1 through 402-m may comprise one or more samples of classifiable data. For example, inputs 402-1 through 402-m may comprise 10 samples of classifiable data. In other embodiments, not all samples of classifiable data may be input into neural network 400.

Neural network 400 may comprise 5 layers of neurons (referred to as layers 404, 406, 408, 410, and 412, respectively corresponding to illustrated nodes 404-1 to 404-m, nodes 406-1 to 406-n, nodes 408-1 to 408-o, nodes 410-1 to 410-p, and node 412). In some embodiments, neural network 400 may have more than 5 layers or fewer than 5 layers. These 5 layers may each be comprised of the same number of neurons as any other layer, more neurons than any other layer, fewer neurons than any other layer, or more neurons than some layers and fewer neurons than other layers. In this embodiment, layer 412 is treated as the output layer. Layer 412 outputs a probability that a target event will occur and contains only one neuron (neuron 412). In other embodiments, layer 412 may contain more than 1 neuron. In this illustration no bias neurons are shown in neural network 400. However, in some embodiments each layer in neural network 400 may contain one or more bias neurons.

Layers 404-412 may each comprise an activation function. The activation function utilized may be, for example, a rectified linear unit (ReLU) function, a SoftPlus function, a Soft step function, or others. Each layer may use the same activation function, but may also transform the input or output of the layer independently of or dependent upon the activation function. For example, layer 404 may be a “dropout” layer, which may process the input of the previous layer (here, the inputs) with some neurons removed from processing. This may help to average the data and can prevent overspecialization of a neural network to one set of data or several sets of similar data. Dropout layers may also help to prepare the data for “dense” layers. Layer 406, for example, may be a dense layer. In this example, the dense layer may process and reduce the dimensions of the feature vector (e.g., the vector portion of inputs 402-1 through 402-m) to eliminate data that is not contributing to the prediction. As a further example, layer 408 may be a “batch normalization” layer. Batch normalization may be used to normalize the outputs of the batch-normalization layer to accelerate learning in the neural network. Layer 410 may be any of a dropout, hidden, or batch-normalization layer. Note that these layers are examples. In other embodiments, any of layers 404 through 410 may be any of dropout, hidden, or batch-normalization layers. This is also true in embodiments with more layers than are illustrated here, or fewer layers.

Layer 412 is the output layer. In this embodiment, neuron 412 produces outputs 414 and 416. Outputs 414 and 416 represent complementary probabilities that a target event will or will not occur. For example, output 414 may represent the probability that a target event will occur, and output 416 may represent the probability that a target event will not occur. In some embodiments, outputs 414 and 416 may each be between 0.0 and 1.0, and may add up to 1.0. In such embodiments, a probability of 1.0 may represent a projected absolute certainty (e.g., if output 414 were 1.0, the projected chance that the target event would occur would be 100%, whereas if output 416 were 1.0, the projected chance that the target event would not occur would be 100%).

In embodiments, FIG. 4 illustrates an example probability-generator neural network with one pattern-recognizer pathway (e.g., a pathway of neurons that processes one set of inputs and analyzes those inputs based on recognized patterns and produces one set of outputs). However, some embodiments may incorporate a probability-generator neural network that may comprise multiple pattern-recognizer pathways and multiple sets of inputs. In some of these embodiments, the multiple pattern-recognizer pathways may be separate throughout the first several layers of neurons, but may merge with another pattern-recognizer pathway after several layers. In such embodiments, the multiple inputs may merge as well. This merger may increase the ability to identify correlations in the patterns identified among different inputs, as well as eliminate data that does not appear to be relevant.

In embodiments, neural network 400 may be trained/adjusted (e.g., biases and weights among nodes may be calibrated) by inputting feedback and/or input to correct/force the neural network to arrive at an expected output. In some embodiments, the feedback may be forced selectively to particular nodes and/or sub-units of the neural network. In some embodiments, the impact of the feedback on the weights and biases may lessen over time, in order to correct for inconsistencies among user(s) and/or datasets. In embodiments, the degradation of the impact may be implemented using a half-life (e.g., the impact degrades by 50% for every time interval of X that has passed) or similar model (e.g., a quarter-life, three-quarter-life, etc.).

It is to be understood that although this disclosure includes a detailed description on cloud computing, implementation of the teachings recited herein are not limited to a cloud computing environment. Rather, some embodiments of the present disclosure are capable of being implemented in conjunction with any other type of computing environment now known or later developed.

Cloud computing is a model of service deliver for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources, but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure, but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities, but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

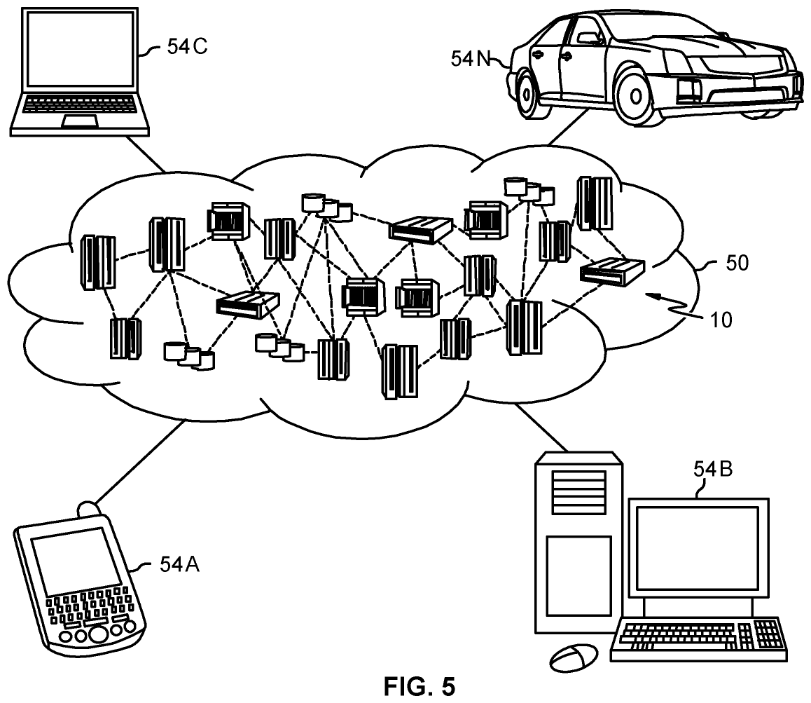

Referring now to FIG. 5, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 10 and cloud computing environment 50 can communicate with any type of computerized device over any type of network and/or network addressable connection (e.g., using a web browser).

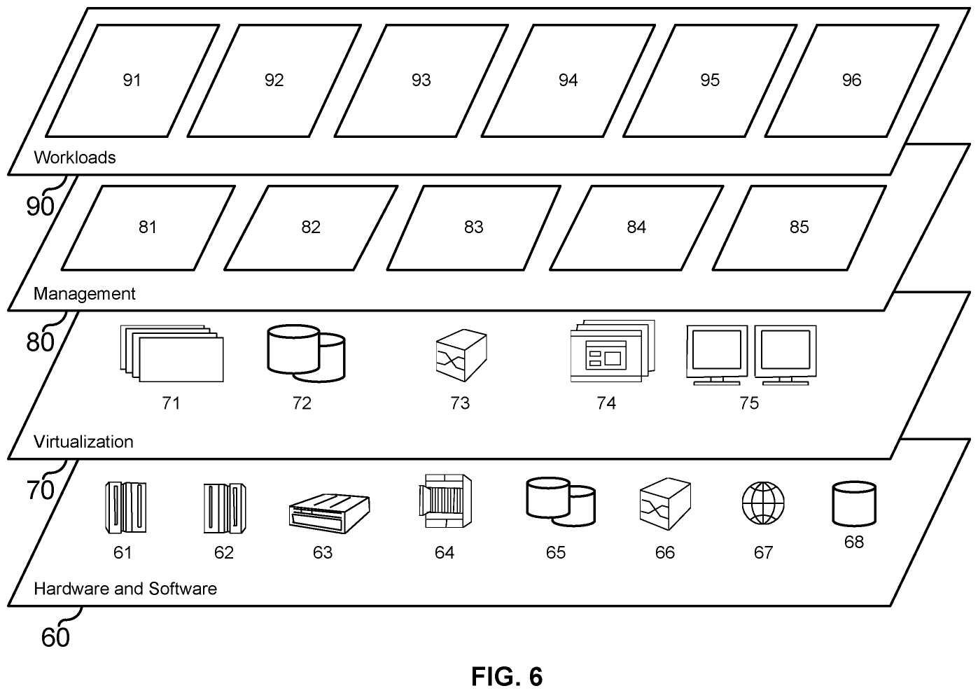

Referring now to FIG. 6, a set of functional abstraction layers provided by cloud computing environment 50 (FIG. 5) is shown. It should be understood in advance that the components, layers, and functions shown in FIG. 6 are intended to be illustrative only and some embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and mitigating treatment risk 96.

Referring now to FIG. 7, shown is a high-level block diagram of an example computer system 701 that may be configured to perform various aspects of the present disclosure, including, for example, method 300, described in FIG. 3. The example computer system 701 may be used in implementing one or more of the methods or modules, and any related functions or operations, described herein (e.g., using one or more processor circuits or computer processors of the computer), in accordance with embodiments of the present disclosure. In some embodiments, the illustrative components of the computer system 701 comprise one or more CPUs 702, a memory subsystem 704, a terminal interface 712, a storage interface 714, an I/O (Input/Output) device interface 716, and a network interface 718, all of which may be communicatively coupled, directly or indirectly, for inter-component communication via a memory bus 703, an I/O bus 708, and an I/O bus interface unit 710.

The computer system 701 may contain one or more general-purpose programmable central processing units (CPUs) 702A, 702B, 702C, and 702D, herein generically referred to as the CPU 702. In some embodiments, the computer system 701 may contain multiple processors typical of a relatively large system; however, in other embodiments the computer system 701 may alternatively be a single CPU system. Each CPU 702 may execute instructions stored in the memory subsystem 704 and may comprise one or more levels of on-board cache. Memory subsystem 704 may include instructions 706 which, when executed by processor 702, cause processor 702 to perform some or all of the functionality described above with respect to FIG. 3.

In some embodiments, the memory subsystem 704 may comprise a random-access semiconductor memory, storage device, or storage medium (either volatile or non-volatile) for storing data and programs. In some embodiments, the memory subsystem 704 may represent the entire virtual memory of the computer system 701 and may also include the virtual memory of other computer systems coupled to the computer system 701 or connected via a network. The memory subsystem 704 may be conceptually a single monolithic entity, but, in some embodiments, the memory subsystem 704 may be a more complex arrangement, such as a hierarchy of caches and other memory devices. For example, memory may exist in multiple levels of caches, and these caches may be further divided by function, so that one cache holds instructions while another holds non-instruction data, which is used by the processor or processors. Memory may be further distributed and associated with different CPUs or sets of CPUs, as is known in any of various so-called non-uniform memory access (NUMA) computer architectures. In some embodiments, the main memory or memory subsystem 704 may contain elements for control and flow of memory used by the CPU 702. This may include a memory controller 705.

Although the memory bus 703 is shown in FIG. 7 as a single bus structure providing a direct communication path among the CPUs 702, the memory subsystem 704, and the I/O bus interface 710, the memory bus 703 may, in some embodiments, comprise multiple different buses or communication paths, which may be arranged in any of various forms, such as point-to-point links in hierarchical, star or web configurations, multiple hierarchical buses, parallel and redundant paths, or any other appropriate type of configuration. Furthermore, while the I/O bus interface 710 and the I/O bus 708 are shown as single respective units, the computer system 701 may, in some embodiments, contain multiple I/O bus interface units 710, multiple I/O buses 708, or both. Further, while multiple I/O interface units are shown, which separate the I/O bus 708 from various communications paths running to the various I/O devices, in other embodiments some or all of the I/O devices may be connected directly to one or more system I/O buses.

In some embodiments, the computer system 701 may be a multi-user mainframe computer system, a single-user system, or a server computer or similar device that has little or no direct user interface, but receives requests from other computer systems (clients). Further, in some embodiments, the computer system 701 may be implemented as a desktop computer, portable computer, laptop or notebook computer, tablet computer, pocket computer, telephone, smart phone, mobile device, or any other appropriate type of electronic device.

It is noted that FIG. 7 is intended to depict the representative example components of an exemplary computer system 701. In some embodiments, however, individual components may have greater or lesser complexity than as represented in FIG. 7, components other than or in addition to those shown in FIG. 7 may be present, and the number, type, and configuration of such components may vary.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for mitigating treatment risk, the method comprising:
receiving a set of baseline treatment information;
determining, from the set of baseline treatment information, a set of incidental information;
generating, based on the set of baseline information and the set of incidental information, a treatment mitigation area;
creating, based on the set of baseline information, the set of incidental information, and the treatment mitigation area, a treatment mitigation model;
identifying, based on the treatment mitigation model, an affected individual; and
providing, for the affected individual, one or more treatment mitigation recommendations.

2. The method of claim 1, further comprising:
monitoring the affected individual;
determining one or more outcomes for the affected individual; and
adjusting, based on the one or more outcomes, the treatment mitigation model.

3. The method of claim 1, wherein the set of baseline treatment information includes a treatment type, a treatment schedule, and a treatment intensity.

4. The method of claim 3, wherein the set of incidental information includes an exposure rate, a total amount of exposure, a grays measurement, and a half-life.

5. The method of claim 3, wherein the set of incidental information includes a clearance rate and a caution score.

6. The method of claim 1, wherein the one or more treatment recommendations is based at least in part on a profile of the affected individual.

7. The method of claim 6, wherein the profile includes at least a set of demographic information for the affected individual and a set of behavioral information for the individual.

8. A computer program product for mitigating treatment risk, the computer program product comprising a computer readable storage medium having program instructions embodied therewith, the program instructions executable by a device to cause the device to:
receive a set of baseline treatment information;
determine, from the set of baseline treatment information, a set of incidental information;
generate, based on the set of baseline information and the set of incidental information, a treatment mitigation area;
create, based on the set of baseline information, the set of incidental information, and the treatment mitigation area, a treatment mitigation model;
identify, based on the treatment mitigation model, an affected individual; and
provide, for the affected individual, one or more treatment mitigation recommendations.

9. The computer program product of claim 8, wherein the program instructions further cause the device to:
monitor the affected individual;
determine one or more outcomes for the affected individual; and
adjust, based on the one or more outcomes, the treatment mitigation model.

10. The computer program product of claim 8, wherein the set of baseline treatment information includes a treatment type, a treatment schedule, and a treatment intensity.

11. The computer program product of claim 10, wherein the set of incidental information includes an exposure rate, a total amount of exposure, a grays measurement, and a half-life.

12. The computer program product of claim 10, wherein the set of incidental information includes a clearance rate and a caution score.

13. The computer program product of claim 8, wherein the one or more treatment recommendations is based at least in part on a profile of the affected individual.

14. The computer program product of claim 13, wherein the profile includes at least a set of demographic information for the affected individual and a set of behavioral information for the individual.

15. A system for mitigating treatment risk, the system comprising:
a memory subsystem, with program instructions included thereon; and
a processor in communication with the memory subsystem, wherein the program instructions cause the processor to:
receive a set of baseline treatment information;
determine, from the set of baseline treatment information, a set of incidental information;
generate, based on the set of baseline information and the set of incidental information, a treatment mitigation area;
create, based on the set of baseline information, the set of incidental information, and the treatment mitigation area, a treatment mitigation model;
identify, based on the treatment mitigation model, an affected individual; and
provide, for the affected individual, one or more treatment mitigation recommendations.

16. The system of claim 15, wherein the program instructions further cause the processor to:
monitor the affected individual;
determine one or more outcomes for the affected individual; and
adjust, based on the one or more outcomes, the treatment mitigation model.

17. The system of claim 15, wherein the set of baseline treatment information includes a treatment type, a treatment schedule, and a treatment intensity.

18. The system of claim 17, wherein the set of incidental information includes an exposure rate, a total amount of exposure, a grays measurement, and a half-life.

19. The system of claim 17, wherein the set of incidental information includes a clearance rate and a caution score.

20. The system of claim 15, wherein the one or more treatment recommendations is based at least in part on a profile of the affected individual.
